(19) United States
(12) Patent Application Publication
(10) Pub. No.: US 2005/0043627 A1
(43) Pub. Date: Feb. 24, 2005

(54) Curved ultrasound transducer arrays manufactured with planar technology

(76) Inventors: Bjorn A.J. Angelsen, Trondheim (NO); Tonni F. Johansen, Trondheim (NO)

Correspondence Address: Lance J. Lieberman, Esq.;Cohen, Pontani, Lieberman & Pavane, Suite 1210, 551 Fifth Avenue, New York, NY, 10176, US

(21) Appl. No.: 10/893,829

(22) Filed: Jul. 19, 2004

Related U.S. Application Data
(60) Provisional application No. 60/487,904, filed on Jul. 17, 2003.

(51) Int. Cl. A61B 8/00; A61B 8/14

(52) U.S. Cl. 600/459

(57) ABSTRACT

A method for manufacturing of ultrasound transducer arrays with a curved surface, where the array is first manufactured on a planar substrate, followed by etching or saw dicing of grooves from the front and/or the back face of the substrate, so that all bending of the substrate occurs along the bottom material of the grooves.

### CROSS REFERENCE TO RELATED APPLICATION

This is application claims priority from U.S. Provisional Patent Application Ser. No. 60/487,404 filed Jul. 17, 2003.

### BACKGROUND OF THE INVENTION

1. Field of the Invention

The present invention is directed to technology and design for efficient manufacturing of ultrasound transducer arrays with a curved array surface. The invention especially addresses manufacturing of arrays with ultrasound frequencies above 10 MHz, and array structures that integrates amplifiers and signal processing electronics close to the array.

2. Description of the Related Art

Medical ultrasound imaging at frequencies above ˜10 MHz, has a wide range of applications for studying microstructures in soft tissues, such as the composition of small tumors or a vessel wall. Due to the increase of ultrasound absorption with frequency, one must for these high frequencies bring the transducers close to the object. This is in the present invention achieved by placing the transducer or transducer array at the distal end of an elongated device such as an endoscope or a catheter, where the distal end is inserted into the body to get the transducer array close to the object, while the proximal end of the elongated device extends outside the body to be connected to an external imaging system.

Capacitive, micromachined ultrasound transducers (cmuts) on silicon is a new and intriguing technique to manufacture transducer arrays at high frequencies. It is especially interesting with this technique that amplifiers, switching circuits, and other processing circuits can be placed on the same Si chip, for compact beam forming with low cost manufacturing.

For the beam forming, curving of the array is desirable in many situations for scanning of the beam according to the switched method or switched synthetic aperture method. However, the manufacturing method for cmut transducers is based on planar technology for silicon processing, which causes a problem for curving of the array.

A similar problem exists with ultrasound arrays made from piezoceramic films on a substrate, such as printing piezoelectric ceramic films onto a planar Si-substrate for example as described in U.S. patent application Ser. No. 10/180,990.

### SUMMARY OF THE INVENTION

The present invention presents a solution to this problem, where the array with connecting electronics first is manufactured on a planar substrate, where etching or saw dicing of grooves from at least one of the faces of the substrate allows the chip to be curved with limited linear strain in the material, so that breaking of the chip in the bending is avoided.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIGS. 1a-1c, show examples of curving of the ultrasound array to obtain three different image formats,

FIGS. 2, shows an ultrasound array with connected amplifiers and beam forming electronics manufactured on a planar substrate.

FIG. 3 shows grooves diced or etched into the substrate surface.

FIG. 4 shows how the strain in the substrate at the groove is related to the curving of the array.

### DETAILED DESCRIPTION OF THE PRESENTLY PREFERRED EMBODIMENTS

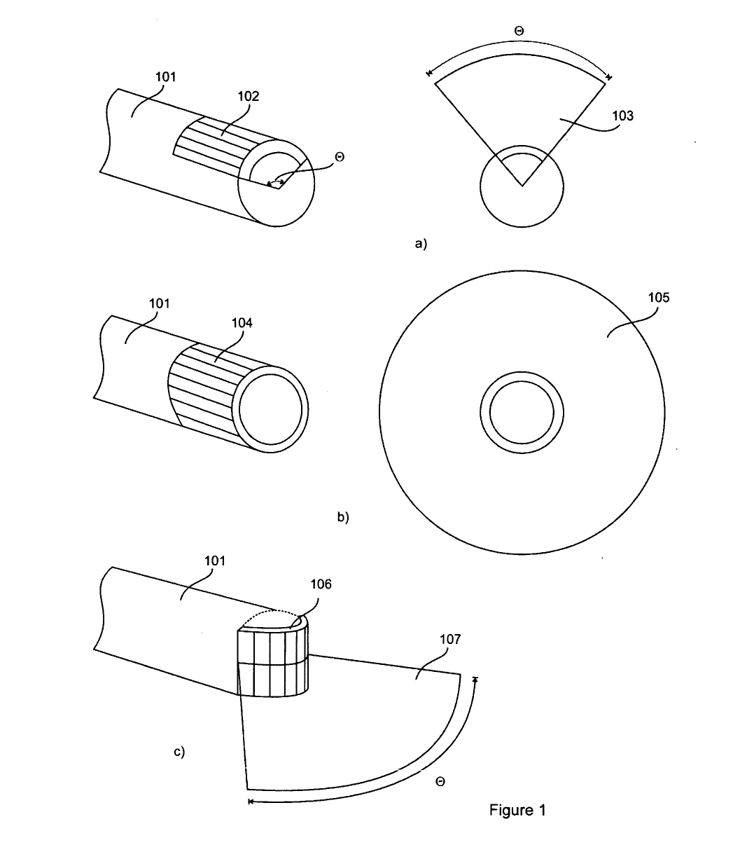

FIG. 1 shows by way of example three typical situations where a curved ultrasound array is mounted to the tip of an elongated device 101, such as a catheter or an endoscope. FIG. 1a shows curving of the ultrasound array 102 around a part of the cylindrical periphery for imaging within a limited sector 103, while FIG. 1b shows curving of the array 104 around the whole periphery of the elongated device for imaging within the full circular cross section 105 around the tip. FIG. 1c shows curving of the array 106 in the forwards direction to the elongated device for imaging in a forward sector 107 of the elongated device 101.

The array is connected to the imaging instrument through a set of wires running along the elongated device. To avoid signal power losses in the wires and maintain a good signal to noise ratio at the higher frequencies (above ˜10 MHz), it is advantageous to place amplifiers on the chip close to the array, so that amplified signals are transmitted on the wires. To minimize the number of wires connecting the array and the imaging instrument, it is further advantageous to apply some beam forming electronics on the Si chip. The simplest form of such electronics is switching transistors for utilizing 1 array element at a time in a sequence along the array, so that synthetic aperture techniques can be applied in the imaging instrument for high resolution image reconstruction. Grouping a set of neighboring elements together and moving the group along the array in steps of one array element, is another interesting beam forming technique that has advantages in signal to noise ratio above the single element synthetic aperture technique. It is further interesting to apply signal delays to the element signals at the chip for electronic focusing and direction steering of the beam.

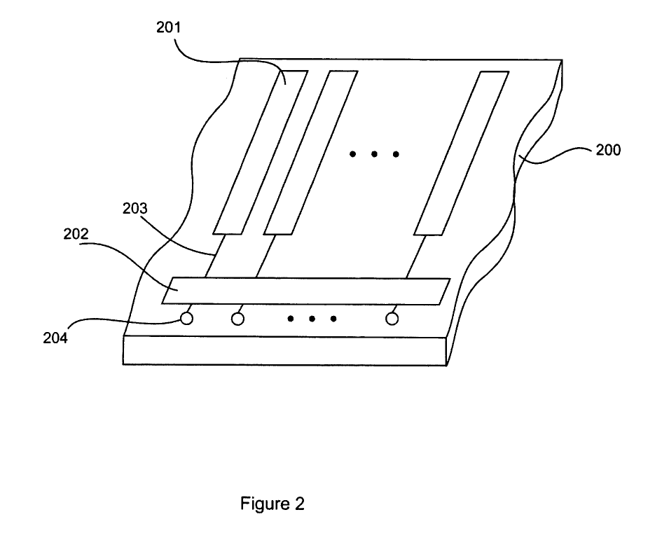

All these procedures are known from prior art, and the essence of this invention is to provide a manufacturing scheme for the ultrasound transducer array and parts of the beam forming electronics that can use planar technology in the first stage manufacturing, with subsequent curving of the array. For this purpose, FIG. 2, shows by way of example a planar Si-chip 200 after the end stage planar processing, where 201 indicates the area of a transducer element, with repeated such elements following in a row to form an array of ultrasound transducer elements. The elements are connected to the amplifier and beam forming electronics 202 through conductors 203 on the chip surface according to standard techniques. The output of the electronics is further connected to bonding islands 204 for connecting to wires that lead the signals through the elongated device to the external instrument.

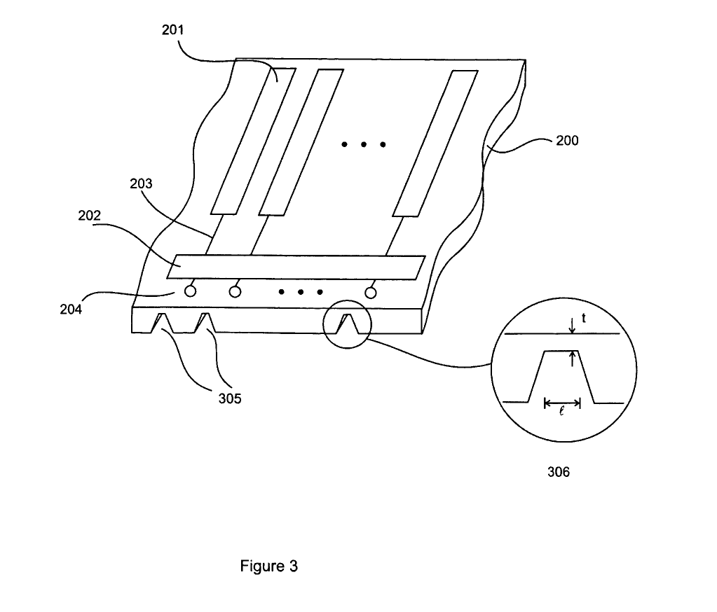

FIG. 3 shows the same chip, where now in addition grooves 305 are etched or diced from the back of the chip between the transducer elements 201. The thickness of the remnant material in the groove is t as illustrated in the magnified drawing 306, where the length of the bottom of the groove is l.

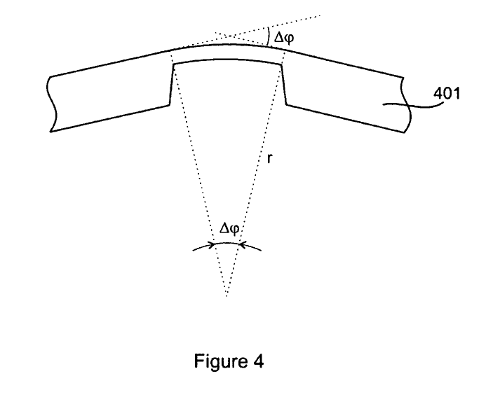

FIG. 4 shows a cross section through the chip 401, where the chip has been bent an angle Δφ along the groove, presenting a radius of curvature r=l/Δφ of the end material in the groove. The maximal strain in the material in the bottom of the groove is with constant bending of the groove equal to

$$ɛ = Δ ϕ \frac{t}{2 l}$$

For example, with N elements around a sector with opening angle Θ as in FIG. 1a and FIG. 1c, we get Δφ=Θ/N. For a full cylinder sector we have as in FIG. 1b Θ=2π. With adequate selections of t, l, and N it is possible to get strains ε<10⁻², which is adequate for bending of a Si-chip around a catheter periphery or alike.

One should also note that for some types of arrays one can etch or dice the grooves from the front side of the chip, and also from both sides of the chip.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A method of manufacturing of curved ultrasound transducer arrays, where
the array is first manufactured on the front side of a planar substrate, and
grooves are etched or diced from the front and/or the back side of the substrate,
the grooves being so deep and wide that sufficient bending of the substrate along the groove bottom can be obtained while the chip between the grooves is approximately flat,
so that the array can be curved for adequate ultrasound beam forming.

2. An ultrasound array according to claim 1, where the array is curved around the whole or parts of the periphery of an elongated device for imaging in a cross section around the distal tip of the device.

3. An ultrasound array according to claim 1, where the array is curved around at the distal tip of an elongated device for imaging in the forwards direction from the

4. An ultrasound array according to claim 1, where the transducer elements are made by printing of ceramic films onto a substrate.

5. An ultrasound array according to claim 1, where the transducer elements are made of capacitive micromachined ultrasound transducers on silicon.

6. An ultrasound array according to claim 4, where signal amplifiers and beam forming circuits are mounted on the same silicon chip.

7. An ultrasound array according to claim 5, where signal amplifiers and beam forming circuits are mounted on the same silicon chip.
